(19) United States
(12) Patent Application Publication
(10) Pub. No.: US 2026/0232330 A1
(43) Pub. Date: Aug. 13, 2026

(54) SUBTALAR ARTHRODESIS NAIL IMPLANT SYSTEM

(71) Applicant: University of Kentucky Research Foundation, Lexington, KY (US)

(72) Inventors: Arun Aneja, Lexington, KY (US); Arjun Srinath, Lexington, KY (US); Gavin Hautala, Pittsville, WI (US); Lorenzo Deveza, Houston, TX (US)

(21) Appl. No.: 19/638,560

(22) Filed: Apr. 3, 2026

Related U.S. Application Data
(62) Division of application No. 18/011,330, filed on Jun. 17, 2021.
(60) Provisional application No. 63/040,038, filed on Jun. 17, 2020.

(51) Int. Cl. A61B 17/17; A61F 2/42; A61B 17/72

(52) U.S. Cl. CPC A61B 17/1725; A61B 17/1775; A61F 2/4202; A61B 17/7225

(57) ABSTRACT

A subtalar nail implant system, an apparatus and spacer for such a subtalar nail implant system and methods of fusing a subtalar joint of a patient allow for primary and revision arthrodesis of the subtalar joint. The system allows for a more stable fusion construct, compression at the fusion site and immediate weight-bearing.

### RELATED APPLICATION

This application claims priority to U.S. Provisional Patent Application Serial No. 63/040,038 filed on June 17, 2020, which is hereby incorporated by reference in its entirety.

### TECHNICAL FIELD

This document relates generally to the field of orthopedic surgery and, more particularly, to a subtalar nail implant system, an apparatus and a spacer for a subtalar nail implant system as well as to methods for fusing the subtalar joint of a patient in order to treat subtalar joint pathology.

### BACKGROUND

The subtalar joint is the articulating surface between the talus and calcaneus in the hindfoot. Injuries of this joint can result from fracture. Pathology can also result from primary osteoarthritis to congenital conditions to post-traumatic sequala, causing loss of function and painful ambulation.

Historically, patients with subtalar pathology can undergo primary arthrodesis (surgical treatment which fuses the talus and calcaneus together) to decrease pain by eliminating movement of the subtalar joint. The current standard of treatment after failed conservative management is to perform subtalar arthrodesis.

The goals of operative treatment are pain relief, deformity correction and functional improvement. This is done by removing the articular cartilage and securing the talus and calcaneus together with two partially threaded, cannulated screws. Current standard of treatment for failed subtalar arthrodesis is to restore hindfoot height and alignment with an allograft or autograft bony block and two screws. This surgical procedure has a high failure rate and requires extensive periods of immobilization. Current nail systems do not allow for restoration of joint height.

This document describes devices and methods that address primary and revision subtalar arthrodesis surgery for arthritic conditions and calcaneal fractures. While current state of the art treatment requires non-weight-bearing on the affected extremity while healing, this implant system and method advantageously allow for a more stable fusion construct, compression at the fusion site, and immediate weight-bearing.

For patients that have failed primary subtalar arthrodesis and require a revision surgery, the present system can restore hindfoot height and apply compression at the fusion site. Custom metal wedges take time to be produced and are cost prohibitive in many cases. The metallic wedge or spacer described in this document will be readily available in multiple sizes. The spacer will be covered in trabeculated titanium to allow for bony integration and fusion. The spacer will be "U-shaped", allowing it to be placed around the nail prior to compression or the nail can be placed through the wedge with a special outrigger.

### SUMMARY

In accordance with the purposes and benefits set forth herein, a subtalar nail implant system is provided. That subtalar nail implant system comprises: (a) a subtalar nail, (b) a spacer adapted to fit in a subtalar joint between a talus and a calcaneus of a patient and (c) a plurality of fasteners to secure the subtalar nail to the talus and to the calcaneus across the subtalar joint of the patient.

In one or more of the many possible embodiments of the subtalar nail implant system, the spacer includes a receiver and the subtalar nail extends through the receiver at the subtalar joint. The spacer includes a body and the receiver is an open channel provided in the body.

In one or more of the many possible embodiments of the subtalar nail implant system, the body of the spacer includes a tapered portion adapted for adjusting spacing between the talus and the calcaneus.

In one or more of the many possible embodiments of the subtalar nail implant system, the tapered portion of the spacer includes a first rough trabecular metal surface for bone ingrowth oriented toward the talus and a second rough trabecular metal face for bone ingrowth oriented toward the calcaneus when in position in the subtalar joint between the talus and the calcaneus. Further, the spacer may include an open lattice section adapted for bone integration.

In one or more of the many possible embodiments of the subtalar nail implant system, the spacer has a substantially U-shaped profile in top plan view.

In one or more of the many possible embodiments of the subtalar nail implant system, the subtalar nail includes a first end, a second end and a plurality of fastener receivers. That plurality of fastener receivers may include (a) a first aperture adjacent the first end and (b) an elongated slot and a second aperture adjacent the second end. The elongated slot may be positioned between the second aperture and the second end.

In one or more of the many possible embodiments of the subtalar nail implant system, the first end of the nail is tapered to aid in insertion into the calcaneus and talus.

In one or more of the many possible embodiments of the subtalar nail implant system, the subtalar nail implant system includes an outrigger having an end adapted for interconnection with the subtalar nail and a plurality of fastener locators adapted for aligning the plurality of fasteners with the plurality of fastener receivers in the subtalar nail.

In one or more of the many possible embodiments of the subtalar nail implant system, the subtalar nail implant system further includes a trial and cooperating guidewire outrigger. When the trial is properly positioned in the subtalar joint, the guidewire outrigger is adapted for placement of a guidewire from the calcaneus into the talus through a guidewire receiver in the trial.

In one or more of the many possible embodiments of the subtalar nail implant system, the trial is shaped like a wedge.

In accordance with an additional aspect, a method is provided for fusing a subtalar joint of a patient. That method includes the steps of; (a) placing a subtalar nail through a calcaneus into a talus of the patient and (b) fixing the subtalar nail to the talus and the calcaneus.

The method may further include the step of placing a spacer around the subtalar nail in the subtalar joint of the patient between the talus and the calcaneus. The method may further include the step of orienting the spacer and the subtalar nail so that the subtalar nail extends through an open ended receiver in the spacer.

The method may further include the step of compressing the subtalar joint.

The method may further include the step of using a medical instrument, such as a lamina spreader, to open the subtalar joint and determine height loss prior to placing the spacer in the subtalar joint.

The method may further include the step of cleaning cartilage and debris from the opened subtalar joint prior to the placing of the spacer in the subtalar joint.

The method may include the step of selecting a spacer of appropriate thickness to properly restore height to the subtalar joint before the placing of the spacer in the subtalar joint.

The method may further include any one or more of the steps of placing a first interlocking screw though the talus into the first aperture to fix the subtalar nail to the talus, placing a second interlocking screw though the calcaneus into the elongated slot to provide compression to the subtalar joint and/or placing a third interlocking screw through the calcaneus and the second aperture to lock the subtalar nail in place and maintain the compression of the subtalar joint.

The method may include the step of positioning a guidewire across the subtalar joint and then drilling and reaming over the guidewire to provide a bore through the calcaneus and into the talus for receiving the subtalar nail before the placing of the subtalar nail in the calcaneus and the talus.

The method may include the step of using an outrigger connected to the subtalar nail to align the first, second and third interlocking screws with the respective first aperture, the elongated slot and the second aperture in the subtalar nail when placing the first, second and third interlocking screws.

In accordance with another aspect, another method of fusing a subtalar joint of a patient comprises the steps of: (a) exposing the subtalar joint, (b) placing a trial and cooperating guidewire outrigger in the exposed subtalar joint, (c) placing a guidewire from the calcaneus to the talus through a guidewire receiver in the trial using the guidewire outrigger as a guide, (d) placing a subtalar nail across the subtalar joint along the path of the guidewire, (e) replacing the trial with a spacer of appropriate thickness to properly restore height to the subtalar joint, (f) compressing the subtalar joint and (g) fixing the subtalar nail to the talus and the calcaneus.

The step of placing the trial and cooperating guidewire outrigger in the exposed subtalar joint may include the steps of cleaning cartilage and other debris from the subtalar joint, using a medical instrument to determine any loss of calcaneal height, selecting a trial of appropriate thickness to restore proper subtalar joint height and broaching the subtalar joint to prepare opposing surfaces of the subtalar joint for correct fit of the trial.

The step of placing the subtalar nail across the subtalar joint may include the steps of: drilling a bore through the calcaneus and into the talus along the guidewire, reaming over the guidewire to prepare the bore to receive the subtalar nail and selecting an appropriate size of subtalar nail for placement within the bore.

The step of fixing the subtalar nail may include the steps of: (a) placing a first interlocking screw though the talus into a first aperture in the subtalar nail to fix the subtalar nail to the talus and placing a second interlocking screw though the calcaneus into an elongated slot in the subtalar nail before the replacing of the trial with the spacer and (b) placing a third interlocking screw through the calcaneus and a second aperture in the subtalar nail to lock the subtalar nail in place and maintain the compression of the subtalar joint after the replacing of the trial with the spacer.

The step of compressing the subtalar joint may include the step of tightening a compression screw within a lumen of the subtalar nail after the replacing of the trial with the spacer and before the placing of the third interlocking screw.

The method may also include the step of using an outrigger connected to the subtalar nail to align the first, second and third interlocking screws with the respective first aperture, the elongated slot and the second aperture in the subtalar nail when placing the first, second and third interlocking screws.

In accordance with yet another aspect, an apparatus for use in fusion of a subtalar joint of a patient, comprises: (a) a trial having a body including a guidewire receiver and (b) a cooperating guidewire outrigger connected to the body and adapted for aligning a guidewire with the guidewire receiver in the body. That apparatus may further include a guidewire whereby when the trial is properly positioned in the subtalar joint, the guidewire extends through the guidewire outrigger, through the calcaneus, through the guidewire receiver in the body within the subtalar joint and into the talus. The body of the apparatus may be at least partially wedge shaped. The guidewire receiver may be an open channel in the body.

In accordance with yet another aspect, a spacer is provided for a subtalar joint. That spacer comprises a body having a receiver adapted to receive a subtalar nail. The body may include a tapered portion adapted for adjusting spacing between a talus and a calcaneus forming the subtalar joint. The tapered portion may include a first rough trabecular metal face oriented toward the talus and a second rough trabecular metal face oriented toward the calcaneus when in position in the subtalar joint between the talus and the calcaneus. The spacer may include an open lattice structure. These rough trabecular metal faces and open lattice structure tend to promote bone ingrowth for integration of the spacer into the now fused subtalar joint.

The receiver in the body of the spacer may be an open channel. The open end of the open channel may be at an edge of the body forming the narrow end of the tapered portion of the body. The body may have a substantially U-shaped profile in plan view.

In the following description, there are shown and described several preferred embodiments of the subtalar nail implant system, the methods of fusing the subtalar joint of a patient, and the apparatus and the spacer for use in the fusion of a subtalar joint. As it should be realized, the system, methods, apparatus and spacer are capable of other, different embodiments and their several details are capable of modification in various, obvious aspects all without departing from the system, method, apparatus and spacer as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the subtalar nail implant system, the apparatus and spacer for such a system and related methods for fusing a subtalar joint of a patient and together with the description serve to explain certain principles thereof.

FIG. 1 is an illustration of the anatomy of the ankle including the talus, the calcaneus and the subtalar joint.

FIG. 2 is a side elevational view of the subtalar nail of the subtalar nail implant system.

FIG. 3 is a detailed view of the internal nail received in the lumen of the subtalar nail of FIG. 2 that is used to provide compression to the subtalar joint.

FIG. 4A is a detailed perspective view of the spacer of the subtalar nail implant system.

FIG. 4B is a side elevation al view of the spacer illustrated in FIG. 4A.

FIG. 5 is a side elevational view illustrating how the outrigger is attached to the second end of the nail and used to align the fasteners or interlocking screws with the fastener receivers provided in the subtalar nail.

FIG. 6 illustrates the subtalar nail implant system properly implanted to as the fuse the subtalar joint of a patient.

FIGS. 7A-7C are respective side elevational, end elevational and top plan views of the trial that is used to (a) determine the size of the spacer required to restore proper height and (b) place the guidewire for the drilling and reaming of the bore through the calcaneus and into the talus for placement the subtalar nail.

FIG. 8A illustrates the placement of the guidewire and preparation for drilling the bore through the calcaneus and into the talus for later placement of the subtalar nail using the trial and cooperating guidewire outrigger.

FIG. 8B illustrates the bore drilled in the calcaneus and the talus for receipt of the subtalar nail.

FIG. 9 illustrates how the outrigger is used to place the interlocking screws into the fastener receivers of the subtalar nail in order to fuse the subtalar joint.

FIGS. 10A-10C are respective side elevational, end elevational and top plan views of a broach used to prepare the subtalar joint for receipt of the trail and subsequent replacement of the trial with the spacer.

Reference will now be made in detail to the present preferred embodiments of the subtalar nail implant system, the apparatus and spacer for such a system and related methods for fusing a subtalar joint of a patient, examples of which are illustrated in the accompanying drawing figures.

### DETAILED DESCRIPTION

Accordingly, it is to be understood that the embodiments of the subtalar nail implant system 10 and related method of fusing the subtalar joint of a patient set forth and described herein are merely illustrative and not restrictive. Reference herein to details of the illustrated embodiments is not intended to limit the scope of the claims. As used herein, the term "and/or" includes "and" and all combinations of one or more of the associated listed items. As used herein, the singular forms "a", "an," and "there" are intended to include the plural forms as well as the singular forms, unless the context clearly indicates otherwise. It will be further understood that the terms "comprises" and/or "comprising," when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this device and method belong. It will be further understood that terms, such as those defined in commonly used dictionaries, should be interpreted as having a meaning that is consistent with their meaning in the context of the relevant art and the present disclosure and will not be interpreted in an idealized overly formal sense unless expressly so defined herein.

In describing the system 10 and method, it will be understood that a number of techniques and steps are disclosed. Each of these have individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of this document and the claims.

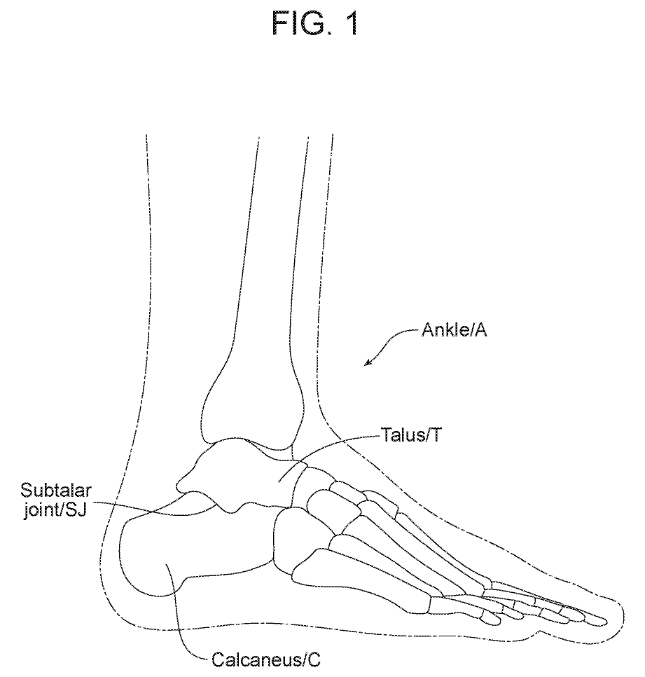

Reference is now made to FIG. 1 which illustrates the anatomy of the human ankle A and clearly shows the bones of the ankle including the talus T and the calcaneus C that form the subtalar joint SJ at their interface. The subtalar nail implant system 10 described in detail in this document is useful in a method of fusing the subtalar joint of a patient in a manner that will be described in detail below.

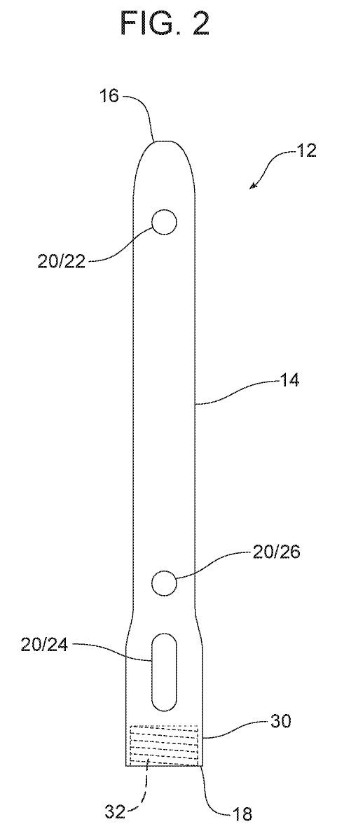

The subtalar nail implant system 10 is illustrated in FIGS. 2-9. The subtalar nail implant system 10 includes a subtalar nail 12 comprising a hollow, elongated body 14 including a rounded or tapered first end 16, a slightly enlarged and an enlarged second end 18. The first end 16 is adapted for insertion into a bore B that is drilled into the calcaneus C and talus T in a manner described in detail below.

As best illustrated in FIGS. 2, the nail 12 includes a plurality of fastener receivers 20 including, particularly, (a) a first aperture 22 adjacent the first end 16 and (b) an elongated slot 24 and a second aperture 26 adjacent the second end 18. In the illustrated embodiment, the elongated slot 24 is provided or positioned between the second aperture 26 and the second end 18.

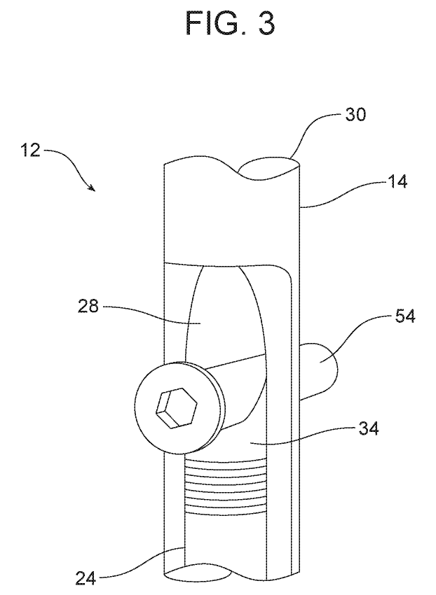

The nail 12 includes a lumen 28 within the tubular wall 30 which includes internal threads 32 at least at the second end 18 thereof (see also FIG. 3). An internal screw 34 within the lumen 28 engages the threads 32 to allow for compression to be applied to the subtalar joint SJ in a manner set forth below (see FIG. 3). The nail 12 may be constructed from implantable stainless steel alloys but could also be constructed of implantable grade titanium alloys, as well. Other material having the requisite properties of strength and inertness may be used.

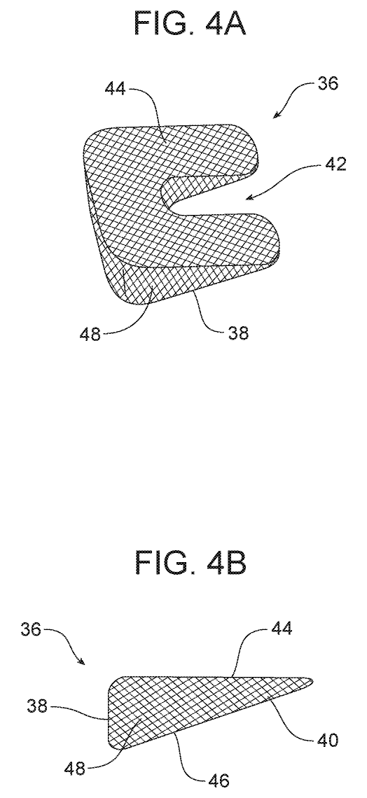

The subtalar nail implant system 10 also includes a spacer 36 adapted to fit in the subtalar joint SJ between the talus T and the calcaneus C (See FIGS. 4A and 4B). The spacer 36 includes a body 38. In the illustrated embodiment, the body 38 includes a tapered portion 40 that acts as a wedge and is adapted for adjusting the spacing between the talus T and the calcaneus C to restore hindfoot height and alignment as necessary for any particular patient.

The spacer 36 also includes a receiver 42 adapted to receive the subtalar nail 12 when the subtalar nail implant system 10 is properly oriented to fuse the subtalar joint SJ of a patient. In the illustrated embodiment, the receiver 42 is an open channel provided in the body. The body 38 has a substantially U-shaped profile in top plan view.

In the illustrated embodiment, the tapered portion 40 includes a first rough trabecular metal surface 44 oriented toward the talus T and a second, rough trabecular surface 46 oriented toward the calcaneus C when the spacer 36 is in position in the subtalar joint SJ between the talus and the calcaneus. The spacer 36 may also include an open lattice section 48. The trabecular metal surfaces 44, 46 and the open lattice section 48 are all adapted to promote bone ingrowth and bone integration. The spacer 36 may be made from materials similar to the nail 12 as described above.

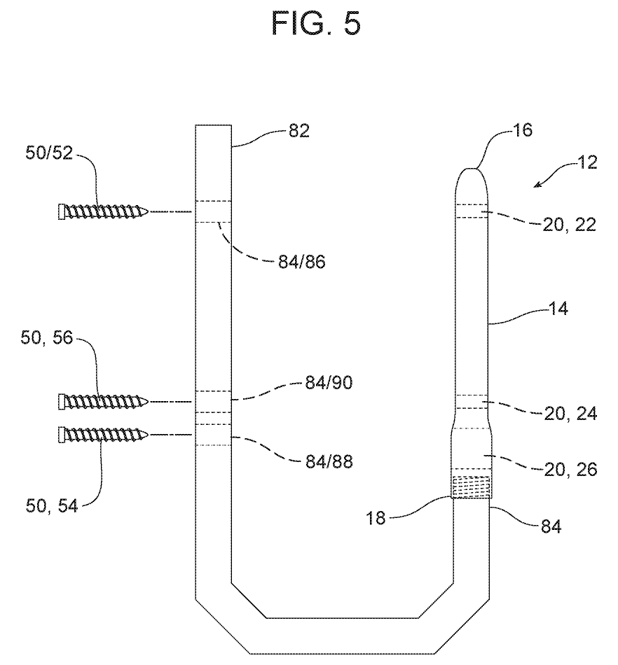

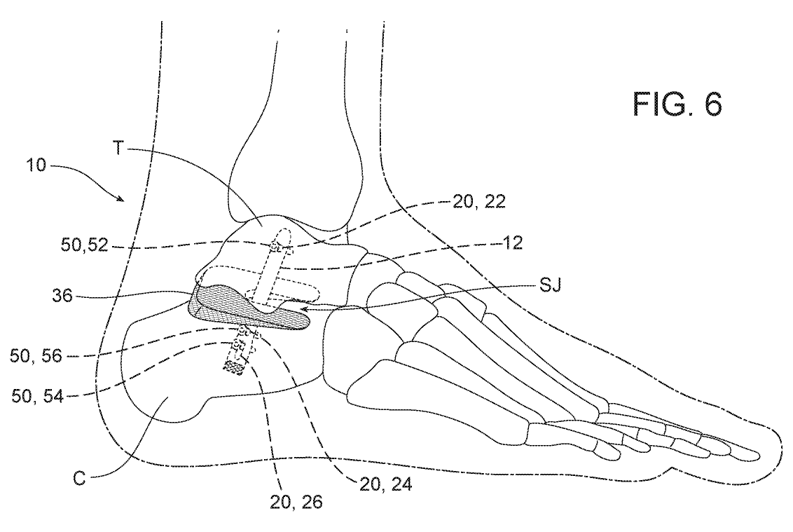

The subtalar nail implant system 10 also includes a plurality of fasteners 50 adapted to secure the subtalar nail 12 to both the talus T and the calcaneus C across the subtalar joint SJ (see FIGS. 5 and 6). Those fasteners 50 in the illustrated embodiment include a first interlocking screw 52 that extends though the talus T into the first aperture 22 in the subtalar nail 12 to fix the subtalar nail to the talus, a second interlocking screw 54 that extends though the calcaneus C into an elongated slot 24 and a third interlocking screw 56 that extends through the calcaneus and the second aperture 26 in the subtalar nail to lock the subtalar nail in place and maintain the compression of the subtalar joint when properly implanted at the subtalar joint SJ (See FIG. 6). The fasteners 50 may be made from materials similar to the nail 12 as described above.

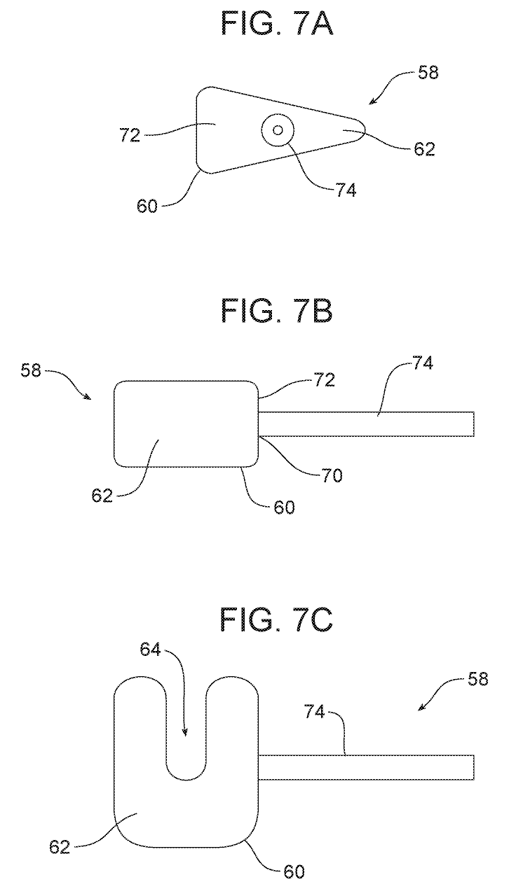

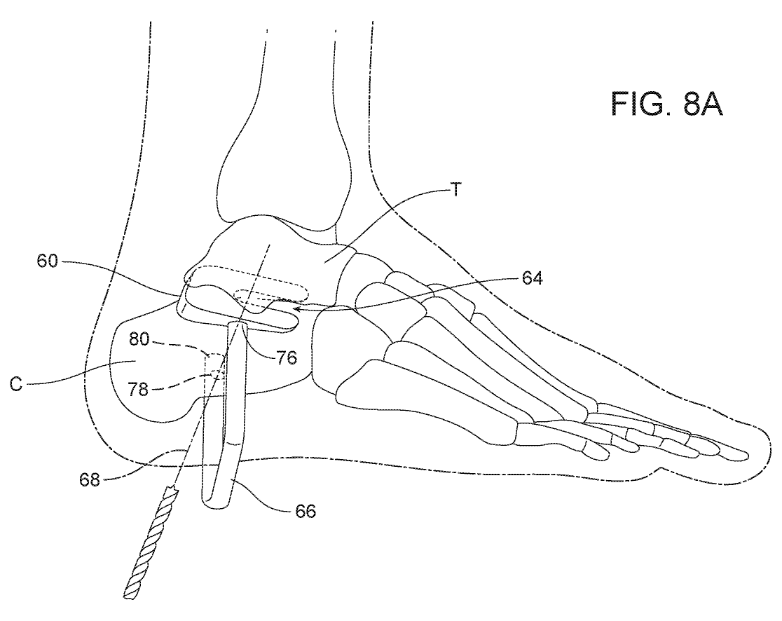

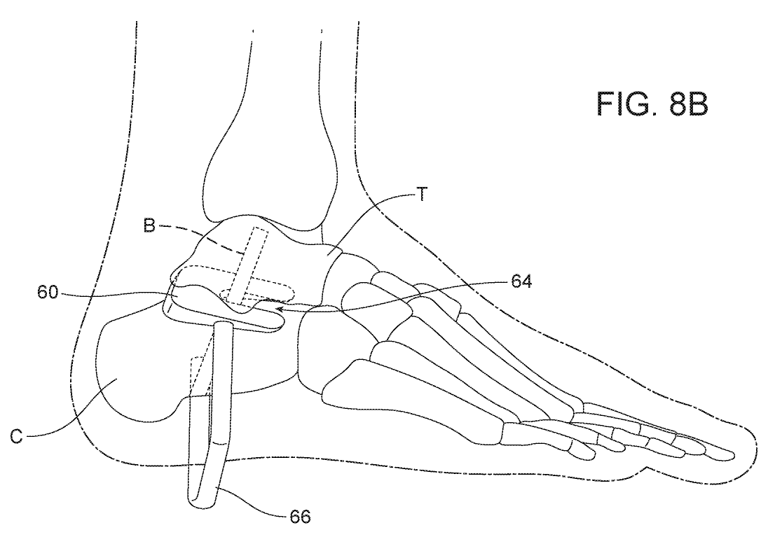

The subtalar nail implant system 10 may also include an apparatus, generally designated by reference numeral 58, that is adapted for properly implanting the nail 12 and spacer 36 to fuse the subtalar joint SJ. See FIGS. 7A-7C, 8A and 8B. That apparatus 58 includes a trial 60 having a body 62 including a guidewire receiver 64. In the illustrated embodiment, the guidewire receiver 64 comprises an open channel. The apparatus 58 also includes a cooperating guidewire outrigger 66 that is connected to the body and adapted for aligning a guidewire 68 with the guidewire receiver 64 in the body 62 when the trial is properly positioned in the subtalar joint SJ.

More specifically, as illustrated in FIGS. 7A-7C, the body 62 is at least partially wedge shaped. The body includes a socket 70 in the sidewall 72 that is adapted to receive an end of a handle 74 that is used to manipulate the trial 60 during placement in the subtalar joint SJ (see FIGS. 8A and 8B). Once the trial 60 is properly placed in the subtalar joint SJ, the handle 74 is removed from the socket 70 and the proximal end 76 of the guidewire outrigger 66 is connected to the socket. The guidewire outrigger 66 includes a guidewire alignment aperture 78 near the distal end 80 of the guidewire outrigger 66 that is adapted to direct (see FIGS. 8A and 8B) a guidewire 68 through the calcaneus C and the guidewire receiver 64 in the body 62 of the trial 60 into the talus T in a manner described in greater detail below. The apparatus 58 may be made from materials similar to the nail 12 as described above.

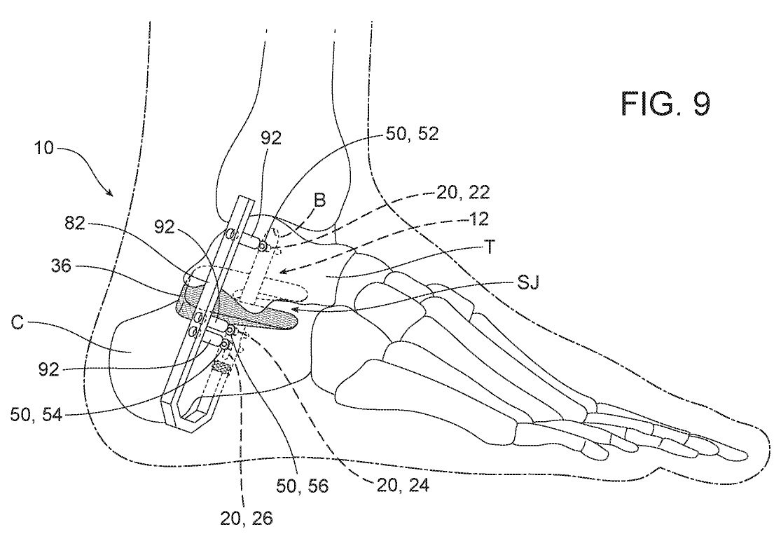

The subtalar nail implant system 10 also includes an outrigger 82 (see FIGS. 5 and 9). In the illustrated embodiment, the outrigger 82 is substantially J-shaped. A distal end 84 of the outrigger 82 is adapted for connection in the second end 18 of the nail 12 by friction fit, threaded connection or other means. The outrigger 82 includes a plurality of fastener locators 84 adapted for aligning the plurality of fasteners 50 with the plurality of fastener receivers 20 in the nail 12. The outrigger 82 may be made from materials similar to the nail 12 as described above.

In the illustrated embodiment, the first fastener locator 86 aligns (a) the first interlocking screw 52 with the first aperture 22, (b) the second fastener locator 88 aligns the second interlocking screw 54 with the elongated slot 24 and the third fastener locator 90 aligns the third interlocking screw 56 with the second aperture 26. A guide sleeve 92 may be inserted into each fastener locator 84 to aid in the placement of the fasteners 50 in a manner known in the art (see US 2020/0113609).

The subtalar nail implant system 10 described above is useful in a method of fusing a subtalar joint SJ of a patient.

Incisions should be thoughtfully planned, and the soft tissues should be handled with care. Joint preparation should be thorough and meticulous, and broad, congruent, bleeding cancellous surfaces should be created, ideally so that apposition of those surfaces can be obtained. All articular cartilage should be removed, as should the subchondral bone. Fixation of the arthrodesis site should be rigid, which this implant system will allow. Particular attention should be paid to the position and alignment of the arthrodesis.

In one possible embodiment of such a method, the spacer 36 is placed at the subtalar joint SJ before the nail 12. Such a procedure may be generally described as follows:

The subtalar joint SJ is approached through a sinus tarsi or posterior lateral vertical incision according to surgeon preference and based on the amount of height restoration needed.

The peroneal tendons are protected and the subtalar joint SJ exposed.

The cartilage is removed and the subtalar joint SJ is prepared.

At this point loss of calcaneal height is determined and the need for the spacer 36 determined.

If there is no loss of height the nail 12 can be placed without the spacer 36.

If the spacer 36 is required the amount of height needed is then determined by placing a medical instrument, such as lamina spreader, into the joint SJ.

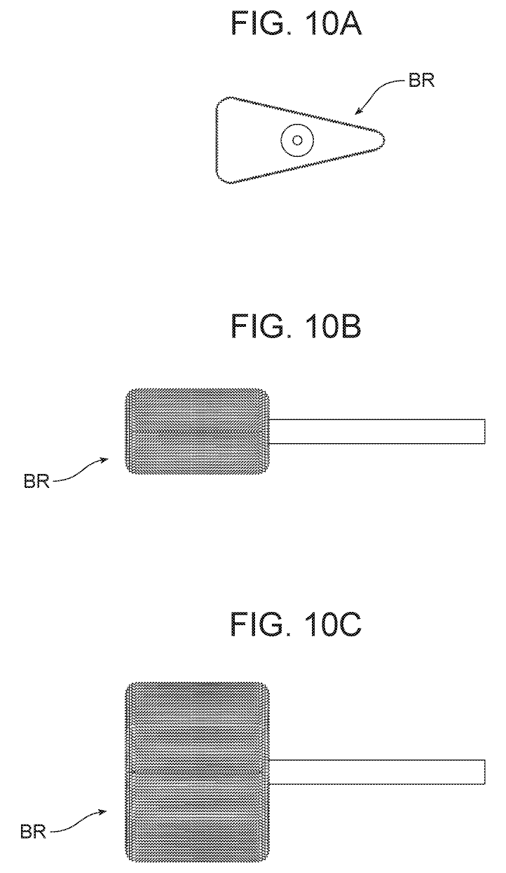

Once the desired height is determined the appropriate broach (see wedge-shaped broach BR illustrated in FIGS. 10A-10C) is used prepare the joint surface ensuring the correct fit.

After broaching, the trial 60 is placed.

The guidewire outrigger 66 connected to the trial 60 allows for placement of the guidewire 68 from the calcaneus C into the talus T through the guidewire receiver 64 in the body 62 of the trial 60.

A drill is then used over the guidewire 68 to drill the calcaneal cortex.

Reaming is performed over the guide wire 68.

The appropriate size subtalar nail 12 is then placed in the bore B in the calcaneus C and the talus T made by the drill: the nail also passing through the guidewire receiver 64 of the trial 60 at the subtalar joint SJ.

A first interlocking screw 52 is placed through the first fastener locator 86 in the outrigger 82 into the talus T and the first aperture 22 in the nail 12.

A second interlocking screw 54 is placed through the second fastener locator 88 in the outrigger 82 through the calcaneus C into the elongated slot 24 in the nail 12.

The trial is removed and then the spacer 36 of proper size is placed around the nail 12 in the subtalar joint SJ.

Compression of the subtalar joint SJ is then provided by tightening the internal screw 34 against the second interlocking screw 54 that extends through the elongated slot 24. Here it should be noted that the elongated slot 24 is elongated along the longitudinal axis of the nail 12 thereby providing clearance for movement of the second interlocking screw and allowing for compression adjustment.

The third interlocking screw 56 is then placed through the third fastener locator 90 in the outrigger 82 through the calcaneus C into the second aperture 26 to lock the nail 12 in place and fuse the subtalar joint SJ.

The outrigger 82 is then removed from the end of the nail 12, the surgical site is made ready for closing and the incision is closed.

In another possible embodiment of such a method, the nail 12 is placed at the subtalar joint SJ before the spacer 36. Such a procedure may be generally described as follows:

Sinus tarsi approach and joint preparation as set forth above.

Guidewire 68 for the nail 12 is placed freehand across the subtalar joint SJ.

Drill over the guidewire 68 to form the bore B in the calcaneus C and talus T that will receive the nail 12.

Ream the bore over the guidewire 68 across the subtalar joint SJ.

Place the nail 12 in the bore B.

A first interlocking screw 52 is placed through the first fastener locator 86 in the outrigger 82 into the talus T and the first aperture 22 in the nail 12.

A second interlocking screw 54 is placed through the second fastener locator 88 in the outrigger 82 through the calcaneus C into the elongated slot 24 in the nail 12.

A trial 60 of proper size for height adjustment is now placed around the nail 12 in the subtalar joint SJ.

The trial is then replaced with a spacer 36 of proper size for height correction.

Compression of the subtalar joint SJ is then provided by tightening the internal screw 34 against the second interlocking screw 54 that extends through the elongated slot 24. Here it should be noted that the elongated slot 24 is elongated along the longitudinal axis of the nail 12 thereby providing clearance for movement of the second interlocking screw and allowing for compression adjustment.

The third interlocking screw 56 is then placed through the third fastener locator 90 in the outrigger 82 through the calcaneus C into the second aperture 26 to lock the nail 12 in place and fuse the subtalar joint SJ.

The outrigger 82 is then removed from the end of the nail 12, the surgical site is made ready for closing and the incision is closed.

In a first possible embodiment, the method of fusing a subtalar joint of a patient may broadly be described as including the steps of; (a) exposing the subtalar joint SJ; (b) placing a trial 60 and cooperating guidewire outrigger 66 in the exposed subtalar joint; (c) placing a guidewire 68 from the calcaneus C to the talus T through a guidewire receiver 64 in the trial using the guidewire outrigger as a guide, (d) placing a subtalar nail 12 across the subtalar joint along the path of the guidewire; (e) replacing the trial with a spacer 36 of appropriate thickness to properly restore height to the subtalar joint; (f) compressing the subtalar joint; and (g) fixing the subtalar nail to the talus and the calcaneus.

The step of placing the trial 60 and cooperating guidewire outrigger 66 in the exposed subtalar joint SJ may include the steps of cleaning cartilage and other debris from the subtalar joint, using a medical instrument, such as a lamina spreader, to determine any loss of calcaneal height, selecting a trial 60 of appropriate height or thickness to restore proper subtalar joint height and broaching the subtalar joint to prepare opposing surfaces of the subtalar joint for correct fit.

The step of placing the subtalar nail 12 across the subtalar joints may include the steps of drilling a bore B through the calcaneus C and into the talus T along the guidewire 68, reaming over the guidewire to prepare the bore to receive the subtalar nail and selecting an appropriate size of subtalar nail for placement within the bore.

The step of fixing the subtalar nail 12 may include the steps of (a) placing a first interlocking screw 52 though the talus T into a first aperture 22 in the subtalar nail to fix the subtalar nail to the talus and placing a second interlocking screw 54 though the calcaneus C into an elongated slot 24 in the subtalar nail before the replacing of the trial 60 with the spacer 36 and (b) placing a third interlocking screw 56 through the calcaneus and a second aperture 26 in the subtalar nail to lock the subtalar nail in place and maintain the compression of the subtalar joint after the replacing of the trial with the spacer.

In this method, the compressing of the subtalar joint SJ may include the tightening of the internal or compression screw 34, within the lumen 28 of the nail 12, against the second interlocking screw 54 after the replacing of the trial 60 with the spacer 36 and before the placing of the third interlocking screw 56.

Still further, the method may include the step of using an outrigger 82 connected to the subtalar nail 12 to align the first, second and third interlocking screws 52, 54, 56 with the respective first aperture 22, the elongated slot 24 and the second aperture 26 in the subtalar nail 12 when placing the first, second and third interlocking screws.

In a second possible embodiment, the method of fusing a subtalar joint of a patient may broadly be described as including the steps of placing a subtalar nail 12 through a calcaneus C into a talus T of the patient and fixing the subtalar nail to the talus and the calcaneus. The subtalar nail 12 may be placed/inserted at substantially any angle traversing the calcaneus C and the talus T. Further, the method may include the step of placing a spacer 36 around the subtalar nail 12 in the subtalar joint SJ of the patient between the talus T and the calcaneus C.

This embodiment of the method may also include the step of orienting the spacer 36 and the subtalar nail 12 so that the subtalar nail extends through an open ended receiver 42 in the spacer. The method may also include one or more of the following steps: (a) compressing the subtalar joint SJ, (b) using a medical instrument, such as a lamina spreader, to open the subtalar joint and determine height loss prior to placing the spacer in the subtalar joint, (c) cleaning cartilage and debris from the opened subtalar joint prior to the placing of the spacer in the subtalar joint, (d) selecting a spacer 36 of appropriate thickness to properly restore height to the subtalar joint before the placing of the spacer in the subtalar joint, (e) placing a first interlocking screw 52 though the talus into the first aperture 22 to fix the subtalar nail to the talus, (f) placing a second interlocking screw 54 though the calcaneus into the elongated slot 24 to provide compression to the subtalar joint, (g) placing a third interlocking screw 56 through the calcaneus and the second aperture 26 to lock the subtalar nail in place and maintain the compression of the subtalar joint, (h) positioning a guidewire 68 across the subtalar joint and then drilling and reaming over the guidewire to provide a bore through the calcaneus and the talus for receiving the subtalar nail before the placing of the subtalar nail in the calcaneus and the talus and (i) using an outrigger connected to the subtalar nail to align the first, second and third interlocking screws with the respective first aperture, the elongated slot and the second aperture in the subtalar nail when placing the first, second and third interlocking screws.

This disclosure may be considered to relate to the following items:

1. A subtalar nail implant system, comprising:

a subtalar nail;

a spacer adapted to fit in a subtalar joint between a talus and a calcaneus of a patient; and

a plurality of fasteners to secure the subtalar nail to the talus and to the calcaneus across the subtalar joint of the patient.

2. The subtalar nail implant system of item 1, wherein the spacer includes a receiver and the subtalar nail extends through the receiver at the subtalar joint.

3. The subtalar nail implant system of item 2, wherein the spacer includes a body and the receiver is an open channel provided in the body.

4. The subtalar nail implant system of item 3, wherein said body includes a tapered portion adapted for adjusting spacing between the talus and the calcaneus.

5. The subtalar nail system of item 4, wherein the tapered portion includes a first rough trabecular metal surface for bone ingrowth oriented toward the talus and a second rough trabecular metal face for bone ingrowth oriented toward the calcaneus when in position in the subtalar joint between the talus and the calcaneus.

6. The subtalar nail implant system of item 5, wherein the spacer includes an open lattice section adapted for bone integration.

7. The subtalar nail implant system of item 6, wherein the subtalar nail includes a plurality of fastener receivers.

8. The subtalar nail implant system of item 7, further including an outrigger having an end adapted for interconnection with the subtalar nail and a plurality of fastener locators adapted for aligning the plurality of fasteners with the plurality of fastener receivers in the subtalar nail.

9. The subtalar nail implant system of item 8, wherein the subtalar nail includes a first end and a second end.

10. The subtalar nail implant system of item 9, wherein the plurality of fastener receivers includes (a) a first aperture adjacent said first end and (b) an elongated slot and a second aperture adjacent said second end.

11. The subtalar nail implant system of item 10, wherein the elongated slot is positioned between the second aperture and the second end.

12. The subtalar nail implant system of item 11, wherein the first end is tapered to aid in insertion into the calcaneus and talus.

13. The subtalar nail implant system of item 12, wherein the spacer has a substantially U-shaped profile in top plan view.

14. The subtalar nail implant system of item 13, further including a trial and cooperating guidewire outrigger whereby when the trial is properly positioned in the subtalar joint, the guidewire outrigger is adapted for placement of a guidewire from the calcaneus into the talus through a guidewire receiver in the trial.

15. The subtalar nail implant system of item 14, wherein the trial is shaped like a wedge.

16. The subtalar nail implant system of any of items 1-12, further including a trial and cooperating guidewire outrigger whereby when the trial is properly positioned in the subtalar joint, the guidewire outrigger is adapted for placement of a guidewire from the calcaneus into the talus through a guidewire receiver in the trial.

17. The subtalar nail implant system of item 16, wherein the trial is shaped like a wedge.

18. The subtalar nail implant system of any of items 1-6, further including an outrigger having an end adapted for interconnection with the subtalar nail and a plurality of fastener locators adapted for aligning the plurality of fasteners with the plurality of fastener receivers in the subtalar nail.

19. The subtalar nail implant system of any of items 1-7, wherein the subtalar nail includes a first end and a second end.

20. The subtalar nail implant system of any of items 1-6, wherein the subtalar nail includes (a) a first aperture adjacent said first end and (b) an elongated slot and a second aperture adjacent said second end.

21. The subtalar nail implant system of claim 20, wherein the elongated slot is positioned between the second aperture and the second end.

22. The subtalar nail implant system of item 21, wherein the first end is tapered to aid in insertion into the calcaneus and talus.

23. The subtalar nail implant system of item 22, wherein the spacer has a substantially U-shaped profile in top plan view.

24. A method of fusing a subtalar joint of a patient, comprising: placing a subtalar nail through a calcaneus into a talus of the patient; and fixing the subtalar nail to the talus and the calcaneus.

25. The method of item 24, further including placing a spacer around the subtalar nail in the subtalar joint of the patient between the talus and the calcaneus.

26. The method of item 25, further including orienting the spacer and the subtalar nail so that the subtalar nail extends through an open ended receiver in the spacer.

27. The method of item 26, further including compressing the subtalar joint.

28. The method of item 27, further including using a medical instrument to open the subtalar joint and determine height loss prior to placing the spacer in the subtalar joint.

29. The method of item 28, including cleaning cartilage and debris from the opened subtalar joint prior to the placing of the spacer in the subtalar joint.

30. The method of item 29, including selecting a spacer of appropriate thickness to properly restore height to the subtalar joint before the placing of the spacer in the subtalar joint.

31. The method of item 30, including placing a first interlocking screw though the talus into the first aperture to fix the subtalar nail to the talus.

32. The method of item 31, including placing a second interlocking screw though the calcaneus into the elongated slot to provide compression to the subtalar joint.

33. The method of item 32, including placing a third interlocking screw through the calcaneus and the second aperture to lock the subtalar nail in place and maintain the compression of the subtalar joint.

34. The method of item 33, including positioning a guidewire across the subtalar joint and then drilling and reaming over the guidewire to provide a bore through the calcaneus and the talus for receiving the subtalar nail before the placing of the subtalar nail in the calcaneus and the talus.

35. The method of item 34, including using an outrigger connected to the subtalar nail to align the first, second and third interlocking screws with the respective first aperture, the elongated slot and the second aperture in the subtalar nail when placing the first, second and third interlocking screws.

36. A method of fusing a subtalar joint of a patient, comprising:

exposing the subtalar joint;

placing a trial and cooperating guidewire outrigger in the exposed subtalar joint;

placing a guidewire from the calcaneus to the talus through a guidewire receiver in the trial using the guidewire outrigger as a guide;

placing a subtalar nail across the subtalar joint along the path of the guidewire;

replacing the trial with a spacer of appropriate thickness to properly restore height to the subtalar joint;

compressing the subtalar joint; and

fixing the subtalar nail to the talus and the calcaneus.

37. The method of item 36, wherein the placing of the trial and cooperating guidewire outrigger in the exposed subtalar joint includes cleaning cartilage and other debris from the subtalar joint, using a medical instrument to determine any loss of calcaneal height, selecting a trial of appropriate thickness to restore proper subtalar joint height and broaching the subtalar joint to prepare opposing surfaces of the subtalar joint for correct fit.

38. The method of item 37, wherein the placing of the subtalar nail across the subtalar joint includes drilling a bore through the calcaneus and into the talus along the guidewire, reaming over the guidewire to prepare the bore to receive the subtalar nail and selecting an appropriate size of subtalar nail for placement within the bore.

39. The method of item 38, wherein the fixing of the subtalar nail includes (a) placing a first interlocking screw though the talus into a first aperture in the subtalar nail to fix the subtalar nail to the talus and placing a second interlocking screw though the calcaneus into an elongated slot in the subtalar nail before the replacing of the trial with the spacer and (b) placing a third interlocking screw through the calcaneus and a second aperture in the subtalar nail to lock the subtalar nail in place and maintain the compression of the subtalar joint after the replacing of the trial with the spacer.

40. The method of item 39, wherein the compressing of the subtalar joint includes tightening a compression screw within a lumen of the subtalar nail against the second interlocking screw after the replacing of the trial with the spacer and before the placing of the third interlocking screw.

41. The method of item 40, including using an outrigger connected to the subtalar nail to align the first, second and third interlocking screws with the respective first aperture, the elongated slot and the second aperture in the subtalar nail when placing the first, second and third interlocking screws.

42. An apparatus for use in fusion of a subtalar joint of a patient, comprising:

a trial having a body including a guidewire receiver; and

a cooperating guidewire outrigger connected to the body and adapted for aligning a guidewire with the guidewire receiver in the body.

43. The apparatus of item 42, further including a guidewire whereby when the trial is properly positioned in the subtalar joint, the guidewire extends along the guidewire outrigger, through the calcaneus, through the guidewire receiver in the body within the subtalar joint and into the talus.

44. The apparatus of item 43, wherein the body is at least partially wedge shaped.

45. The apparatus of item 44, wherein the guidewire receiver is an open channel in the body.

46. A spacer for a subtalar joint, comprising a body having a receiver adapted to receive a subtalar nail.

47. The spacer of item 46, wherein the body includes a tapered portion adapted for adjusting spacing between a talus and a calcaneus forming the subtalar joint.

48. The spacer of item 47, wherein the tapered portion includes a rough trabecular metal face oriented toward the talus and a second rough trabecular metal face oriented toward the calcaneus when in position in the subtalar joint between the talus and the calcaneus.

49. The spacer of any of items 46-48, wherein the spacer includes an open lattice section adapted for bone integration.

50. The spacer of item 49, wherein the receiver is an open channel in the body.

51. The spacer of item 50, wherein the open channel has an open end at an edge of the body forming a narrow end of the tapered portion.

52. The spacer of item 51, wherein the body has a substantially U-shaped profile in top plan view.

53. The spacer of any of items 46-48, wherein the receiver is an open channel in the body.

54. The spacer of item 53, wherein the open channel has an open end at an edge of the body forming a narrow end of the tapered portion.

55. The spacer of item 54, wherein the body has a substantially U-shaped profile in top plan view.

56. The spacer of any of items 46-48, wherein the body has a substantially U-shaped profile in top plan view.

Terms of approximation, such as the terms about, substantially, approximately, etc., as used herein, refers to ± 10 % of the stated numerical value. Use of the terms parallel or perpendicular are meant to mean approximately meeting this condition, unless otherwise specified.

It is to be fully understood that certain aspects, characteristics, and features, of the subtalar nail implant system 12, the spacer 36, the apparatus 58 and the method, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the subtalar nail implant system 12, the spacer 36, the apparatus 58 and the method which are illustratively described and presented in combination or sub-combination in the context or format of a single embodiment may also be illustratively described and presented in the context or format of a plurality of separate embodiments.

Although the subtalar nail implant system 12, the spacer 36, the apparatus 58 and the method of this disclosure have been illustratively described and presented by way of specific exemplary embodiments, and examples thereof, it is evident that many alternatives, modifications, or/and variations, thereof, will be apparent to those skilled in the art. Accordingly, it is intended that all such alternatives, modifications, or/and variations, fall within the spirit of, and are encompassed by, the broad scope of the appended claims.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

What is claimed:

1. A subtalar nail implant system, comprising:
a subtalar nail;
a spacer adapted to fit in a subtalar joint between a talus and a calcaneus of a patient; and
a plurality of fasteners to secure the subtalar nail to the talus and to the calcaneus across the subtalar joint of the patient.

2. The subtalar nail implant system of claim 1, wherein the spacer includes a receiver and the subtalar nail extends through the receiver at the subtalar joint.

3. The subtalar nail implant system of claim 2, wherein the spacer includes a body and the receiver is an open channel provided in the body.

4. The subtalar nail implant system of claim 3, wherein said body includes a tapered portion adapted for adjusting spacing between the talus and the calcaneus.

5. The subtalar nail system of claim 4, wherein the tapered portion includes a first rough trabecular metal surface for bone ingrowth oriented toward the talus and a second rough trabecular metal surface for bone ingrowth oriented toward the calcaneus when in position in the subtalar joint between the talus and the calcaneus.

6. The subtalar nail implant system of claim 5, wherein the spacer includes an open lattice section adapted for bone integration.

7. The subtalar nail implant system of claim 6, wherein the subtalar nail includes a plurality of fastener receivers.

8. The subtalar nail implant system of claim 7, further including an outrigger having an end adapted for interconnection with the subtalar nail and a plurality of fastener locators adapted for aligning the plurality of fasteners with the plurality of fastener receivers in the nail.

9. The subtalar nail implant system of claim 8, wherein the subtalar nail includes a first end and a second end.

10. The subtalar nail implant system of claim 9, wherein the plurality of fastener receivers includes (a) a first aperture adjacent said first end and (b) an elongated slot and a second aperture adjacent said second end.

11. The subtalar nail implant system of claim 10, wherein the elongated slot is positioned between the second aperture and the second end.

12. The subtalar nail implant system of claim 11, wherein the first end is tapered to aid in insertion into the calcaneus and talus.

13. The subtalar nail implant system of claim 12, wherein the spacer has a substantially U-shaped profile in top plan view.

14. The subtalar nail implant system of claim 13, further including a trial and cooperating guidewire outrigger whereby when the trial is properly positioned in the subtalar joint, the guidewire outrigger is adapted for placement of a guidewire from the calcaneus into the talus through a guidewire receiver in the trial.

15. The subtalar nail implant system of claim 14, wherein the trial is shaped like a wedge.

16. The subtalar nail implant system of any of claims claim 1 - 12, further including a trial and cooperating guidewire outrigger whereby when the trial is properly positioned in the subtalar joint, the guidewire outrigger is adapted for placement of a guidewire from the calcaneus into the talus through a guidewire receiver in the trial.

17. The subtalar nail implant system of claim 16, wherein the trial is shaped like a wedge.

18. The subtalar nail implant system of any of claims claim 1 - 6, further including an outrigger having an end adapted for interconnection with the subtalar nail and a plurality of fastener locators adapted for aligning the plurality of fasteners with the plurality of fastener receivers in the subtalar nail.

19. The subtalar nail implant system of any of claims claim 1 - 7, wherein the subtalar nail includes a first end and a second end.

20. The subtalar nail implant system of any of claims claim 1 - 6, wherein the subtalar nail includes (a) a first aperture adjacent said first end and (b) an elongated slot and a second aperture adjacent said second end.

21. The subtalar nail implant system of claim 20, wherein the elongated slot is positioned between the second aperture and the second end.

22. The subtalar nail implant system of claim 21, wherein the first end is tapered to aid in insertion into the calcaneus and talus.

23. The subtalar nail implant system of claim 22, wherein the spacer has a substantially U-shaped profile in top plan view.

24. A method of fusing a subtalar joint of a patient, comprising:
placing a subtalar nail through a calcaneus into a talus of the patient; and
fixing the subtalar nail to the talus and the calcaneus.

25. The method of claim 24, further including placing a spacer around the subtalar nail in the subtalar joint of the patient between the talus and the calcaneus.

26. The method of claim 25, further including orienting the spacer and the subtalar nail so that the subtalar nail extends through an open ended receiver in the spacer.

27. The method of claim 26, further including compressing the subtalar joint.

28. The method of claim 27, further including using a medical instrument to open the subtalar joint and determine height loss prior to placing the spacer in the subtalar joint.

29. The method of claim 28, including cleaning cartilage and debris from the opened subtalar joint prior to the placing of the spacer in the subtalar joint.

30. The method of claim 29, including selecting a spacer of appropriate thickness to properly restore height to the subtalar joint before the placing of the spacer in the subtalar joint.

31. The method of claim 30, including placing a first interlocking screw though the talus into the first aperture to fix the subtalar nail to the talus.

32. The method of claim 31, including placing a second interlocking screw though the calcaneus into the elongated slot to provide compression to the subtalar joint.

33. The method of claim 32, including placing a third interlocking screw through the calcaneus and the second aperture to lock the subtalar nail in place and maintain the compression of the subtalar joint.

34. The method of claim 33, including positioning a guidewire across the subtalar joint and then drilling and reaming over the guidewire to provide a bore through the calcaneus and the talus for receiving the subtalar nail before the placing of the subtalar nail in the calcaneus and the talus.

35. The method of claim 34, including using an outrigger connected to the subtalar nail to align the first, second and third interlocking screws with the respective first aperture, the elongated slot and the second aperture in the subtalar nail when placing the first, second and third interlocking screws.

36. A method of fusing a subtalar joint of a patient, comprising:
exposing the subtalar joint;
placing a trial and cooperating guidewire outrigger in the exposed subtalar joint;
placing a guidewire from the calcaneus to the talus through a guidewire receiver in the trial using the guidewire outrigger as a guide;
placing a subtalar nail across the subtalar joint along the path of the guidewire;
replacing the trial with a spacer of appropriate thickness to properly restore height to the subtalar joint;
compressing the subtalar joint; and
fixing the subtalar nail to the talus and the calcaneus.

37. The method of claim 36, wherein the placing of the trial and cooperating guidewire outrigger in the exposed subtalar joint includes cleaning cartilage and other debris from the subtalar joint, using a medical instrument to determine any loss of calcaneal height, selecting a trial of appropriate thickness to restore proper subtalar joint height and broaching the subtalar joint to prepare opposing surfaces of the subtalar joint for correct fit.

38. The method of claim 37, wherein the placing of the subtalar nail across the subtalar joint includes drilling a bore through the calcaneus and into the talus along the guidewire, reaming over the guidewire to prepare the bore to receive the subtalar nail and selecting an appropriate size of subtalar nail for placement within the bore.

39. The method of claim 38, wherein the fixing of the subtalar nail includes (a) placing a first interlocking screw though the talus into a first aperture in the subtalar nail to fix the subtalar nail to the talus and placing a second interlocking screw though the calcaneus into an elongated slot in the subtalar nail before the replacing of the trial with the spacer and (b) placing a third interlocking screw through the calcaneus and a second aperture in the subtalar nail to lock the subtalar nail in place and maintain the compression of the subtalar joint after the replacing of the trial with the spacer.

40. The method of claim 39, wherein the compressing of the subtalar joint includes tightening a compression screw within a lumen of the subtalar nail against the second interlocking screw after the replacing of the trial with the spacer and before the placing of the third interlocking screw.

41. The method of claim 40, including using an outrigger connected to the subtalar nail to align the first, second and third interlocking screws with the respective first aperture, the elongated slot and the second aperture in the subtalar nail when placing the first, second and third interlocking screws.

42. An apparatus for use in fusion of a subtalar joint of a patient, comprising:
a trial having a body including a guidewire receiver; and
a cooperating guidewire outrigger connected to the body and adapted for aligning a guidewire with the guidewire receiver in the body.

43. The apparatus of claim 42, further including a guidewire whereby when the trial is properly positioned in the subtalar joint, the guidewire extends along the guidewire outrigger, through the calcaneus, through the guidewire receiver in the body within the subtalar joint and into the talus.

44. The apparatus of claim 43, wherein the body is at least partially wedge shaped.

45. The apparatus of claim 44, wherein the guidewire receiver is an open channel in the body.

46. A spacer for a subtalar joint, comprising a body having a receiver adapted to receive a subtalar nail.

47. The spacer of claim 46, wherein the body includes a tapered portion adapted for adjusting spacing between a talus and a calcaneus forming the subtalar joint.

48. The spacer of claim 47, wherein the tapered portion includes a rough trabecular metal face oriented toward the talus and a second rough trabecular metal face oriented toward the calcaneus when in position in the subtalar joint between the talus and the calcaneus.

49. The spacer of any of claims claim 46 - 48, wherein the spacer includes an open lattice section adapted for bone integration.

50. The spacer of claim 49, wherein the receiver is an open channel in the body.

51. The spacer of claim 50, wherein the open channel has an open end at an edge of the body forming a narrow end of the tapered portion.

52. The spacer of claim 51, wherein the body has a substantially U-shaped profile in top plan view.

53. The spacer of any of claims claim 46 - 48, wherein the receiver is an open channel in the body.

54. The spacer of claim 53, wherein the open channel has an open end at an edge of the body forming a narrow end of the tapered portion.

55. The spacer of claim 54, wherein the body has a substantially U-shaped profile in top plan view.

56. The spacer of any of claims claim 46 - 48, wherein the body has a substantially U-shaped profile in top plan view.
